(19) United States
(12) Patent Application Publication
(10) Pub. No.: US 2007/0191715 A1
(43) Pub. Date: Aug. 16, 2007

(54) Method and device for the in vivo observation with embedded cell and tissue

(76) Inventor: Hiroyuki Yonekawa, Bethlehem, PA (US)

Correspondence Address: Hiroyuki Yonekawa, 3500 Corporate PKW, Olympus America/ SEG, Center Valley, PA, 18034-0610, US

(21) Appl. No.: 11/698,966

(22) Filed: Jan. 29, 2007

Related U.S. Application Data
(60) Provisional application No. 60/763,206, filed on Jan. 30, 2006.

(51) Int. Cl. A61B 6/00

(52) U.S. Cl. 600/476

(57) ABSTRACT

This invention is cited to the method and device for the In Vivo observation of embedded cells or tissues. Using this device, we can observe cells or tissues in inside of living animal for long period with high quality of images. We can couple this device with electro-physiological devices or drug administration devices too. According to this invention, biological reactions of target cells or tissues in inside of animal are observed in real time. Then, arrayed multiplex form of device is also claimed. Such arrayed multiplex device works as high throughput analyzing device In Vitro as well as In Vivo. This device can be used for the drug screening and drug susceptibility test by adding second layer. According to this invention, multiple biological reactions as metastasis of cancer, immunological reaction, drug resistance or host-parasite interaction of target cells can be analyzed at once.

### BACKGROUND OF INVENTION

In vivo analysis technique is widely used in biological research field. But still it is difficult to observe cellular function within the living animal. X-ray, CT, MRI (Magnetic Resonance Imaging) or optical techniques are used for this purpose.

Then, the methods of implanting of artificial material and optical devices in inside of animal are investigated in animal research as well as clinical treatment (Ref.1).

A lot of physiological and imaging technologies are applied in live animal research (Ref. 2). Practical methods and devices of embedded cells or tissue offer straightforward approach in experimental animal (Ref 3). And coupled measurement method offers real time in vivo type experiments (Ref 4).

Organ compatible materials as silicon or collagen matrix are widely used in clinical and biological applications (Ref 5). Optimized observing devices for embedded cells or tissues are strongly investigated. And specialized three-dimensional conformational analysis device was developed in reference 6.

### SUMMARY OF THE INVENTION

This invention is concerned to the cell and tissue analysis method in vivo in claim 1-6. This invention contains physiological sensing technique coupled with this observation device in claim 2. And this device can be combined with passive experimental device as electric stimuli or microinjecting device in claim 3 and 4. Also this invention includes a flexible tube, which accepts thin optics, instead of light guide as an alternative observing device in claim 5.

This invention also contains implantable arrayed matrix device in claim 6. Embedded cell or tissue can be observed by optical device as microscope in claim 7. Further we can analyze the 3D morphology of cells or tissues by sheared force 3-D observation device in claim 8. At last, this invention includes the multiple cells or tissue culturing method using arrayed matrix device in claim 9. If we apply second layer of matrix for each cubicles, we can obtain multiple analysis results for each cells or tissues at different cubicles in vitro or in vivo at once.

This invention provides simple and efficient cells and tissue analysis method and device. Then this invention offers accurate cellular or tissue response in vitro as well as in vivo. This device can achieve complex biological assay. Also parallel analysis with multiplex reactions can run altogether.

### EMBODIMENT 1 (FIG. 1)

The device is composed with two major parts. The first part is a permeable shell or capsule (1). This capsule (1) contains cells or tissue (2) inside as embedded culture medium or gelling medium as Matrigel (M, Ref 5). This capsule works as incubator for the cells or tissue in inside of animal. Embedding cells or tissue in inside of animal is prepared by ordinal laborites technique as ref. 3. Depend on the pore size or permeable shell (1), we can choose variety of biological reaction as pharmaceutical application (small chemicals size as 1000d MW) to immunological reaction (Immunoglobline size as 150 kd MW). The second part (2) is the optical window for the observation. Precisely designed lens, optical fiber or grin lens can be used for this purpose. Both surface (2A, 2B) have enough optical quality and this part (2) can transmit the image of cells or tissues into outside. Then, cellular or tissue images can be observed by imaging device (D) from outside of animal (A). By attaching this capsule (1) to the optical window (2) directly, the influence of vibration of heartbeat or respiration of animal does not affect the image. So stable image can be obtained by imaging device (D)

### EMBODIMENT 2 (FIG. 2)

The device is composed with three major parts. The first part (1) and second part (2) are same as embodiment 1. The third part (3) is the electrode for the measuring electrical potential of cells, tissue or microenvironment (M).

Then we can observe the image of cells or tissues with obtaining physiological signals at same time by this combination device (AD).

### EMBODIMENT 3 (FIG. 3)

The device is composed with three major parts. The first part (1) and second part (2) are same as embodiment. The third part (4) is the electrode or microinjection device. Electrode works for the electrical stimulating of cells or tissues. Microinjection device works for the administrating drug or reagents into the cells or tissues (M). Then we can observe the biological reaction with such stimuli with the image of cells or tissue at once by this combination (SD).

### EMBODIMENT 4 (FIG. 4)

The device is composed with two major arts. The first part (1) is same as embodiment 1. The second part (5) is composed by flexible tube as lubber. The second part (5) close after embedding the device in animal, but can accept the optical measuring device as fiberscope (6) for the observing cells or tissue (M). Precisely designed lens, optical fiber or grin lens can be used for this purpose.

### EMBODIMENT 5 (FIG. 5)

The device is composed with four major parts. The first part is composed with solid matrix as glass or plastic for segregating cells or tissues in inside of device (7). This matrix (7) forms arrayed cubicles for sustaining cells or tissues (8) in inside of each cubicles. Cells or tissues (8) in such cubicles are embedded in the gelling materials as gelatin, fibrin, collagen, agarose or Matrigel (M, Ref 4). These materials hold cells or tissue under the innate condition as well as works for the optical windows. The third and 4th parts are permeable matrix or membrane for the transferring the nutrient or chemical compound for embedded cells or tissue (9 and 10). By the nature of permeability and biological compatibility, embedded cells or tissues (8) can alive and response to their environment as intact cells. We can apply the gradient or orientation of chemicals according to the usage of asymmetric permeability of material for part (9) and part (10). Varieties of biological reactions are monitored at once using different cell types or tissue types in each cubicle. Ordinal microscope can be used in case of in vitro observation. And if we apply in vivo observation of this device, we need to use special intravital microscope for observing cells or tissue, IV100, Olympus America Inc, Center valley, Pa. 18034 (USA). Dimensions of each layer are optimized according to the size of animal.

### EMBODIMENT 6 (FIG. 6)

This matrix device is composed with five major parts. First four components are similar to the embodiment 5. Only the second layer of the permeable matrix (9) have compartment for suppression of dispersing chemicals into next cubicles and contains different chemicals. Then, non-permeable clear cover forms the protecting layer (11). Then, we can observe the cellular responses or tissue responses against to the each chemical in layer (7) by microscope (in vitro) or Intravital microscope (in vivo)

### EMBODIMENT 7 (FIG. 7)

The matrix device is transferred into the 3-D observing chamber (12), which is cited in Ref. 3. According to their flexible nature of matrix, we can observe real 3-D morphology of each cells or tissues under the microscope.

### EMBODIMENT 8 (FIG. 8)

We can incubate this device in Petri dish (13A) and inside of animal (13B). Then, we can observe the cellular responses or tissue responses against to the each chemical in layer (7) by microscope (13A) or Intravital microscope (13B)

### FIGURES

FIG. 1: A schematic cross-section of device and its use.

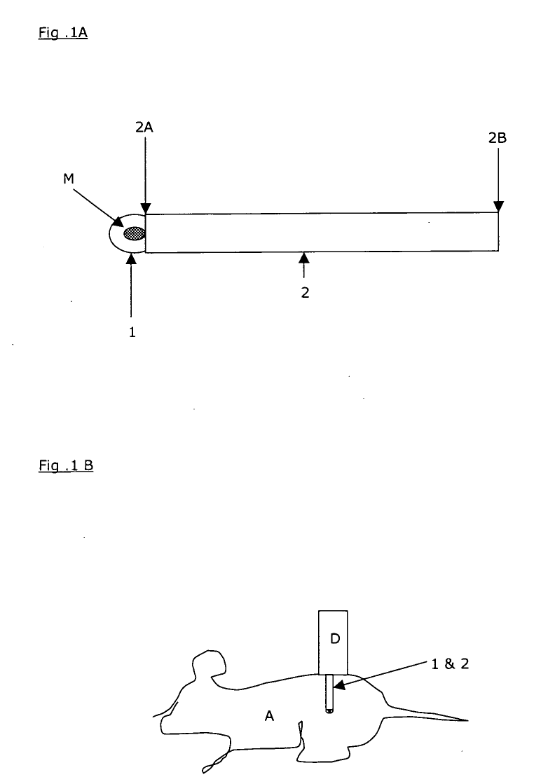

FIG. 2: A schematic cross-section of device and its use with physiological sensing device.

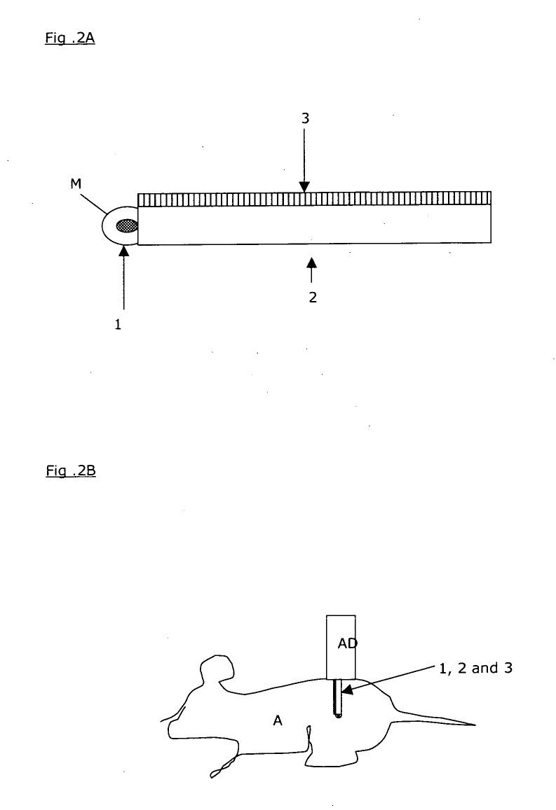

FIG. 3: A schematic cross-section of device and its use with physiological stimulating device or microinjecting device.

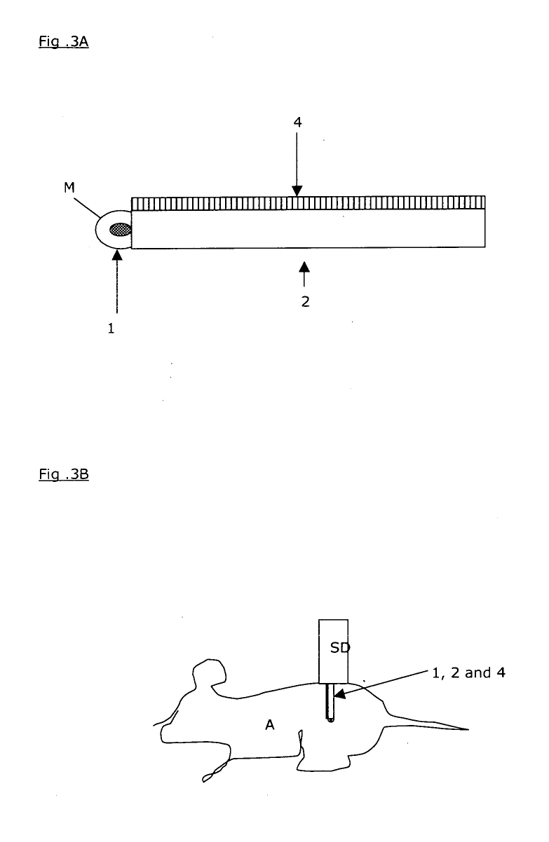

FIG. 4: A schematic cross-section of device and its use.

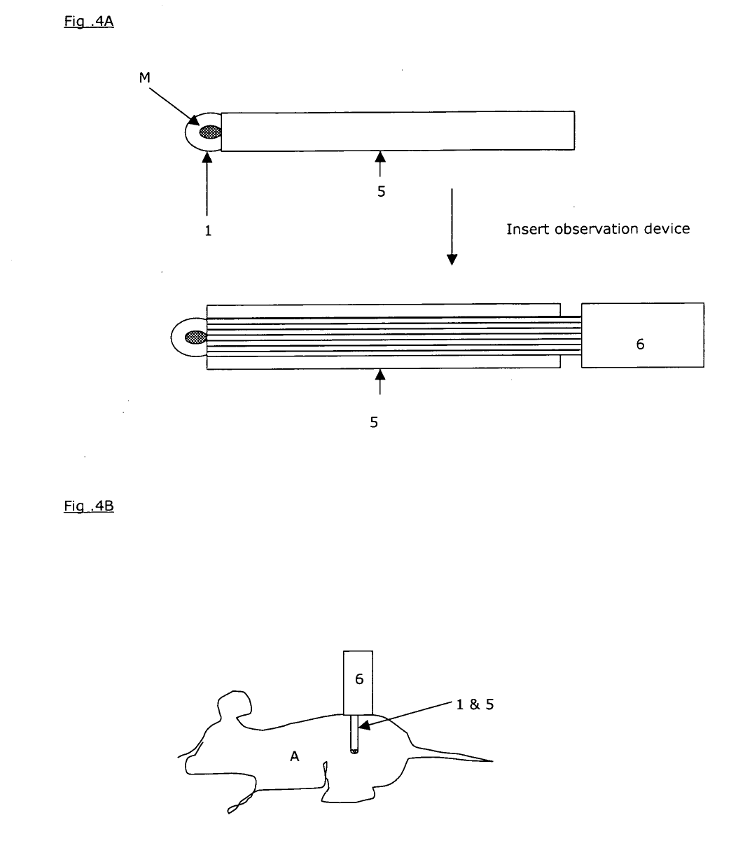

FIG. 5: A schematic illustration of matrix device and in use.

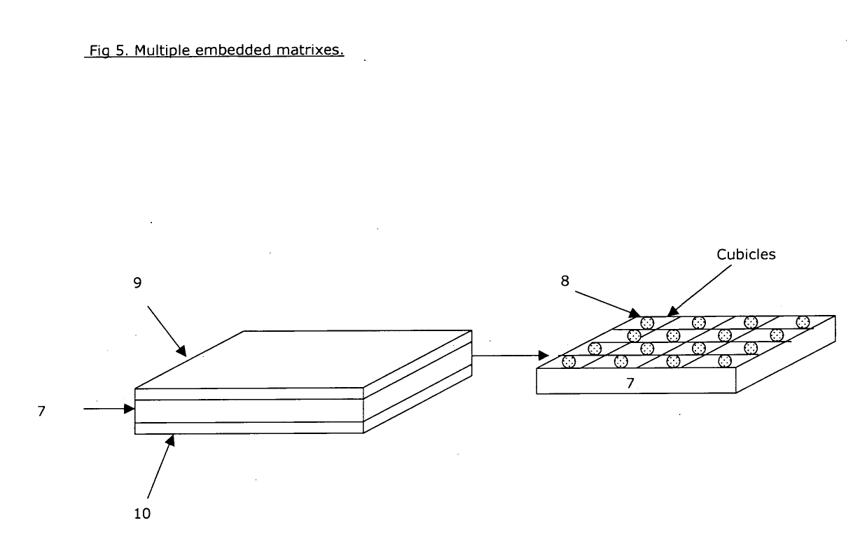

FIG. 6: A schematic illustration of drug screening or susceptible test case.

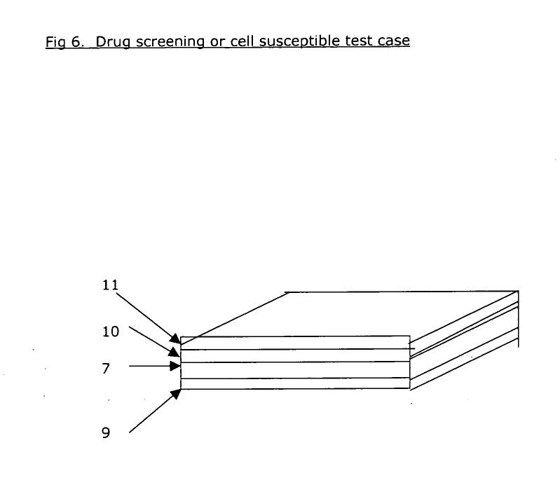

FIG. 7: A schematic illustration of 3D analysis.

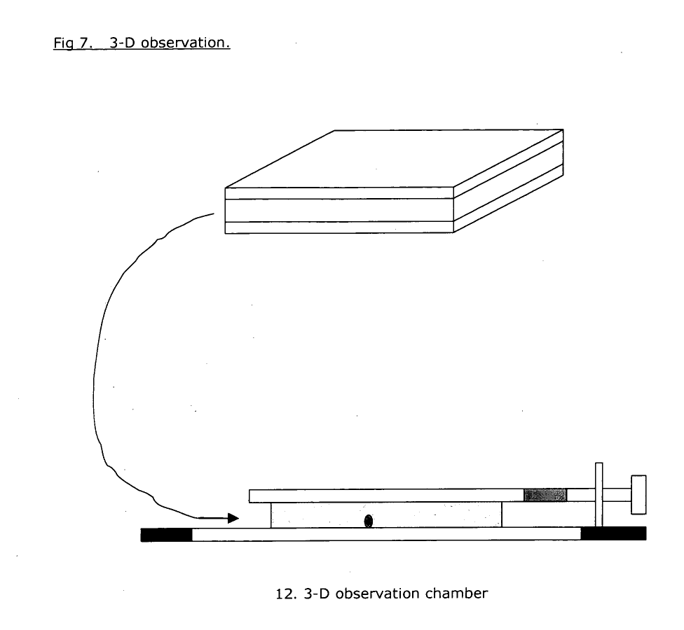

FIG. 8: A schematic illustration of culturing device in vitro (13A) and in vivo (13B).

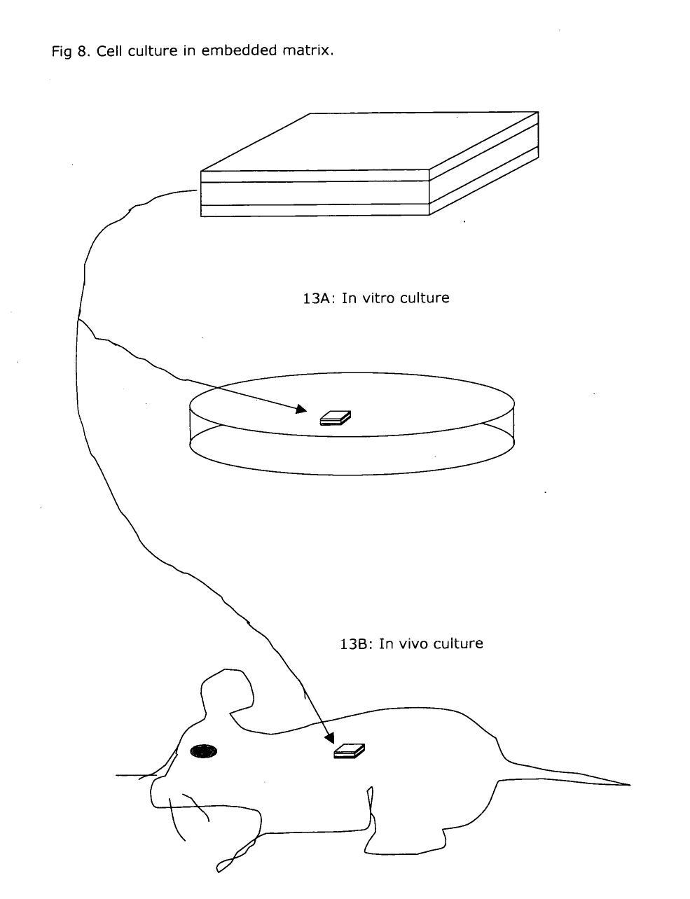

### REFERENCES

Ref 1: U.S. Pat. No. 6,470,124 and US patent application: 20050237604
Ref 2: Looking and listing to light. By R. Weissleder, et.al. Nature method, PP313-31, vol 23, Year of 2005.
Ref 3: Okino M, et.al. Jpn J Cancer Res, (1987) 78: 1319-1321
Ref 4:Alginated encapsulation is a highly reproducible method for tumor cell implantation in dorsal skin fold chambers, Yong Wang, Qing Chem and Fan Yuan, BioTechniques 39:834-839 (December, 2005)
Ref 5: BD Bioscience, catalogue: B04G044 & B04B045. Two Oak Park, Bedford, Mass. 01730 (USA)
Ref 6: US patent application: 11/438,028

1. Analytical method and device for an In Vivo observation method with embedded cells or tissue in permeable capsule with light guide.

2. The analytical method and device, which is coupled with physiological measuring device with embedded cells or tissue in permeable capsule.

3. The analytical method and device, which is coupled with physiological stimulating device with embedded cells or tissue in permeable capsule.

4. The analytical method and device, which is coupled with the microinjecting device with embedded cells or tissue in permeable capsule.

5. The analytical method and device, which has flexible tube instead of light guide with embedded cells or tissue in permeable capsule.

6. Arrayed and implantable matrix device for embedding cells or tissues.

7. The analytical method and device for observing arrayed and implantable matrix device.

8. Three-dimensional analysis with arrayed and implantable matrix device.

9. Cell culturing method by the arrayed and implantable matrix device.
